(19) United States
(12) Patent Application Publication
(10) Pub. No.: US 2014/0373648 A1
(43) Pub. Date: Dec. 25, 2014

(54) SYSTEM AND METHOD FOR AUTOMATIC RECORDING OF A PLURALITY OF MEASUREMENTS AND VERIFICATION OF SPECIMENS IN ROCK MECHANICS

(71) Applicants: Natasa Stambuk Cvitanovic, Split (HR); Predrag Dukic, Split (HR)

(72) Inventors: Natasa Stambuk Cvitanovic, Split (HR); Predrag Dukic, Split (HR)

(21) Appl. No.: 14/304,602

(22) Filed: Jun. 13, 2014

Related U.S. Application Data
(60) Provisional application No. 61/836,938, filed on Jun. 19, 2013.

(51) Int. Cl. G01N 3/06; G01B 11/14; G01B 21/20; G01N 33/24

(52) U.S. Cl. CPC G01N 3/066; G01N 33/24; G01B 11/14; G01B 21/20
USPC 73/865.8

(57) ABSTRACT

A system and method for automatic recording of a plurality of measurements and verification of specimens in rock mechanics is disclosed. The system may include a server system, a support surface, a motorized stage, a plate, a V-block, one or more digital dial gauges, a measurement system controller, a slide motion controller and a moving frame. The method may include the steps of securing the rock specimens onto a V-block of a system to automatically record a plurality of measurements and verify rock specimens, actuating a linear motorized stage to collect a plurality of rock specimens data, sliding a digital dial gauge tip along one or more of a plurality of chosen lines at the same time on the specimen surface, transmitting a plurality of chosen surface lines data to the system to automatically record a plurality of measurements and verify rock specimens and recording the chosen surface lines data.

This application claims priority to U.S. Provisional Application 61/836,938 filed on Jun. 19, 2013, the entire disclosure of which is incorporated by reference.

### BACKGROUND OF THE INVENTION

1. Field of the Invention

The present invention is a system and method. More specifically, the present invention is a system and method for automatic recording of a plurality of measurements and verification of specimens in rock mechanics or more briefly a system and method for automatic recording and verification of rock specimens. A plurality of measurements is referring to a plurality of simultaneous shape and surface measurements on the cylindrical test specimens prepared from rock core. Specimens in rock mechanics or rock specimens is referred to test specimens of intact rock planned for further laboratory testing of strength and deformability properties.

### BRIEF SUMMARY OF THE INVENTION

The present invention is a system and method. More specifically, the present invention is a system and method for automatic recording of a plurality of measurements and verification of specimens in rock mechanics.

The system to automatically record a plurality of measurements and verify rock specimens includes a server system with a processor system, a communications interface, a communications system, an input system and an output system, the server system having access to a communications network, a communications module, a web browser module, a web server application and a non-transitory storage media, the processor system is in communication with the memory system via the communications system. The system may also include a support surface, a motorized stage, a plate, a V-block, a plurality of digital or other dial gauge tips, a measurement system controller, a slide motion controller and a moving frame.

The method for automatic recording of a plurality of measurements and verification of rock specimens includes securing the rock mechanics specimens onto a V-block of a system to automatically record a plurality of measurements and verify rock specimens, actuating a linear motorized stage to collect a plurality of rock specimens data, sliding a digital dial gage tip (by movement of the specimen) along one or more of a plurality of chosen lines at the same time on the specimen surface, transmitting a plurality of chosen surface lines data to a server system and recording the chosen surface lines data.

It is an object of the present invention to provide a system and method for automatic recording and verification of rock specimens that is intended for preliminary testing of intact rock specimens for rock mechanics studies in geotechnics, civil engineering and mining and solves the problem of effective and reliable recording of cylindrical specimens and their shape deviations.

It is an object of the present invention to provide a system and method for automatic recording and verification of rock specimens that improves testing according to ASTM D4543, which traditionally has been a time consuming and relatively inefficient process.

It is an object of the present invention to provide a system and method for automatic recording and verification of rock specimens that significantly improves quality and reduces time and cost of the testing by automatically and simultaneously recording a number of surface profiles along a plurality of chosen lines on the sides and the ends of a rock specimen.

It is an object of the present invention to provide a system and method for automatic recording and verification of rock specimens that provides information regarding ASTM D4543 not found as published in the field such as from scientific databases.

It is an object of the present invention to provide a system and method for automatic recording and verification of rock specimens that replaces a manual process with a plurality of automatic and discrete readings with continuous curved-surface and wavelike profiles.

It is an object of the present invention to provide a system and method for automatic recording and verification of rock specimens where a combination of mechanical and electrical components with automatic data acquisition and processing of a plurality of measurements is designed and proven in a laboratory.

It is an object of the present invention to provide a system and method for automatic recording and verification of rock specimens where a user selects a plurality of recording lines.

It is an object of the present invention to provide a system and method for automatic recording and verification of rock specimens that after recording is completed, all the necessary parameters are instantly calculated such as side straightness, flatness, parallelism and perpendicularity of the specimen ends.

### BRIEF DESCRIPTION OF THE DRAWINGS

The present invention will be described by way of exemplary embodiments, but not limitations, illustrated in the accompanying drawing in which like references denote similar elements, and in which:

FIG. 1 illustrates a system overview of a system to automatically record a plurality of measurements and verify one or more rock specimens, in accordance with one embodiment of the present invention.

FIG. 2 illustrates a block diagram of a server system, in accordance with one embodiment of the present invention.

FIG. 3 illustrates a block diagram of a client system, in accordance with one embodiment of the present invention.

FIG. 4 illustrates a graph of a manually recorded surface profile along a diameter (to be replaced with an automatically recorded surface profile), in accordance with one embodiment of the present invention.

FIG. 5 illustrates a device to manually record a surface profile along a diameter on the specimen end for the purpose of verification of rock specimen (to be replaced with a device to automatically record a surface profile), in accordance with one embodiment of the present invention.

FIG. 6 illustrates a graph of an automatically recorded surface profile along a diameter on a specimen end, in accordance with one embodiment of the present invention.

FIG. 7 illustrates a graph of one or more surface profiles along a plurality of straight lines on a specimen cylindrical surface, in accordance with one embodiment of the present invention.

FIG. 8 illustrates a system overview of a plurality of system components to automatically record a plurality of measurements and verify rock specimens, in accordance with one embodiment of the present invention.

### DETAILED DESCRIPTION OF ILLUSTRATIVE EMBODIMENTS

Various aspects of the illustrative embodiments will be described utilizing terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention. However, the order of description should not be construed as to imply that these operations are necessarily order dependent. In particular, these operations need not be performed in the order of presentation.

The phrase “in one embodiment” is utilized repeatedly. The phrase generally does not refer to the same embodiment, however, it may. The terms “comprising”, “having” and “including” are synonymous, unless the context dictates otherwise.

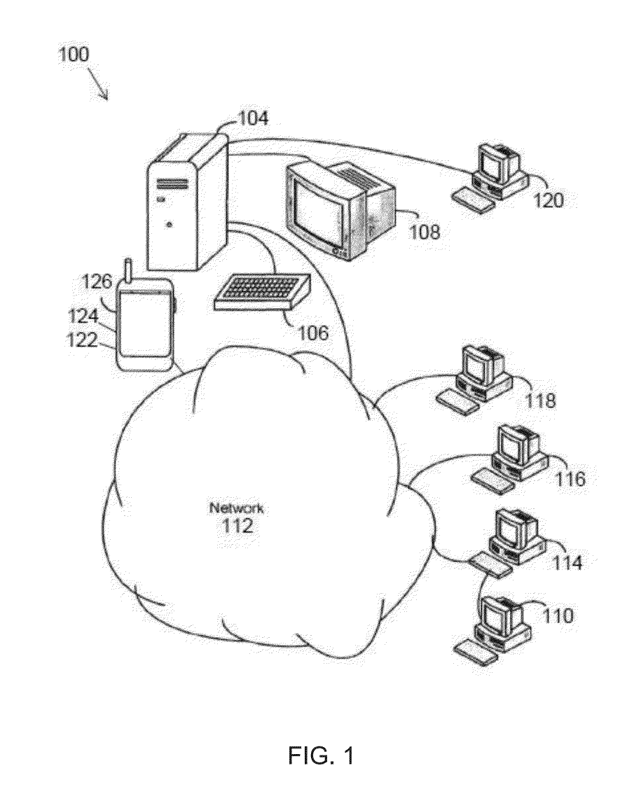

FIG. 1 illustrates a system overview of a system 100 to automatically record a plurality of measurements and verify one or more rock specimens, in accordance with one embodiment of the present invention.

The system 100 may include a server system 104, an input system 106, an output system 108, a plurality of client systems 110, 114, 116, 118 and 120, a communications network 112 and a handheld or mobile device 122. In other embodiments, the system 100 may include additional components and/or may not include all of the components listed above.

The server system 104 may include one or more servers. One server 104 may be the property of the distributor of any related software or non-transitory storage media. In other embodiments, the system 100 may include additional components and/or may not include all of the components listed above.

The input system 106 may be utilized for entering input into the server system 104, and may include any one of, some of, any combination of, or all of a keyboard system, a mouse system, a track ball system, a track pad system, a plurality of buttons on a handheld system, a mobile system, a scanner system, a wireless receiver, a microphone system, a connection to a sound system, and/or a connection and/or an interface system to a computer system, an intranet, and/or the Internet (i.e., IrDA, USB).

The output system 108 may be utilized for receiving output from the server system 104, and may include any one of, some of, any combination of or all of a monitor system, a wireless transmitter, a handheld display system, a mobile display system, a printer system, a speaker system, a connection or an interface system to a sound system, an interface system to one or more peripheral devices and/or a connection and/or an interface system to a computer system, an intranet, and/or the Internet.

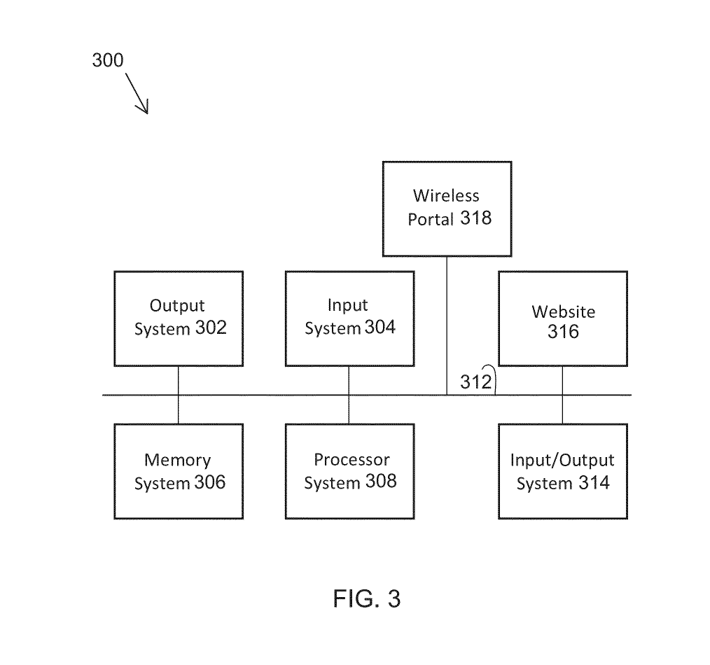

The system 100 may illustrate some of the variations of the manners of connecting to the server system 104, which may be a website (FIG. 3, 316) such as an information providing website or other suitable type of website. The server system 104 may be directly connected and/or wirelessly connected to the plurality of client systems 110, 114, 116, 118 and 120 and may be connected via the communications network 112. Client systems 120 may be connected to the server system 104 via the client system 118. The communications network 112 may be any one of, or any combination of, one or more local area networks or LANs, wide area networks or WANs, wireless networks, telephone networks, the Internet and/or other networks. The communications network 112 may include one or more wireless portals. The client systems 110, 114, 116, 118 and 120 may be any system that an end user may utilize to access the server system 104. For example, the client systems 110, 114, 116, 118 and 120 may be personal computers, workstations, tablet computers, laptop computers, game consoles, hand-held network enabled audio/video players, mobile devices and/or any other network appliance.

The client system 120 may access the server system 104 via the combination of the communications network 112 and another system, which may be the client system 118. The client system 120 may be a handheld or a mobile wireless device 122, such as a mobile phone, a tablet computer or a handheld network-enabled audio/music player, which may also be utilized for accessing network content. The client system 120 may be a cell phone with an operating system or SMARTPHONE® 124 or a tablet computer with an operating system or IPAD® 126 or other suitable client system.

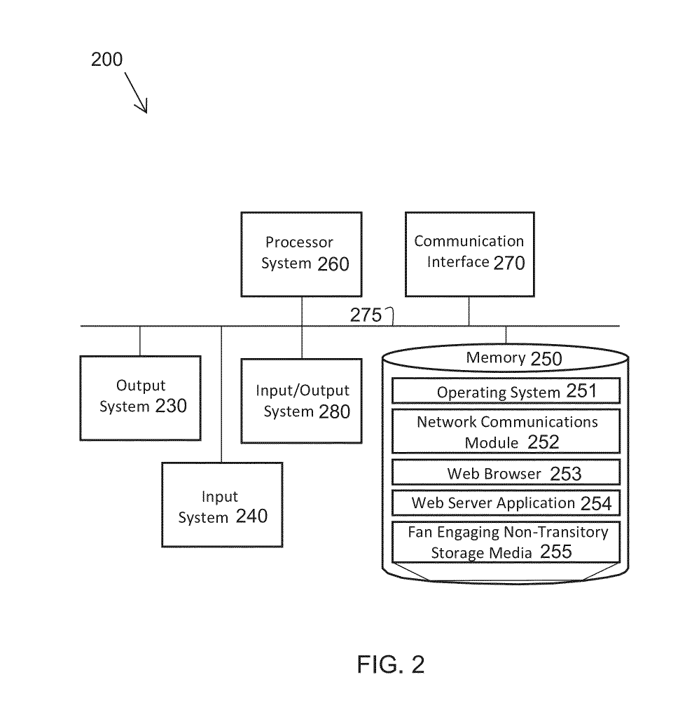

FIG. 2 illustrates a block diagram of a server system 200, in accordance with one embodiment of the present invention.

The server system 200 may include an output system 230, an input system 240 and a memory system 250, which may store an operating system 251, a communications module 252, a web browser module 253, a web server application 254 and a rock specimens recording and verifying non-transitory storage media 255. The server system 200 may also include a processor system 260, a communications interface 270, a communications system 275 and an input/output system 280. In other embodiments, the server system 200 may include additional components and/or may not include all of the components listed above.

The output system 230 may include any one of, some of, any combination of, or all of a monitor system, a handheld display system, a printer system, a speaker system, a connection or an interface system to a sound system, an interface system to one or more peripheral devices and/or a connection and/or interface system to a computer system, an intranet, and/or the Internet.

The input system 240 may include any one of, some of, any combination of, or all of a keyboard system, a mouse system, a track ball system, a track pad system, one or more buttons on a handheld system, a scanner system, a microphone system, a connection to a sound system, and/or a connection and/or an interface system to a computer system, an intranet, and/or the Internet (i.e., IrDA, USB).

The memory system 250 may include any one of, some of, any combination of, or all of a long term storage system, such as a hard drive; a short term storage system, such as a random access memory; or a removable storage system, such as a floppy drive or a removable drive and/or a flash memory. The memory system 250 may include one or more machine readable mediums that may store a variety of different types of information. The term machine readable medium may be utilized to refer to any medium capable of carrying information that may be readable by a machine. One example of a machine-readable medium may be a computer-readable medium such as a non-transitory storage media. The memory system 250 may store one or more machine instructions for rock specimens recording and verifying. The operating system 251 may control all software or non-transitory storage media and hardware of the system 100. The communications module 252 may enable the input system 304 to communicate on the communications network 112. The web browser module 253 may allow for browsing the Internet. The web server application 254 may serve a plurality of web pages to client systems that request the web pages, thereby facilitating browsing on the Internet.

The processor system 260 may include any one of, some of, any combination of, or all of multiple parallel processors, a single processor, a system of processors having one or more central processors and/or one or more specialized processors dedicated to specific tasks. The processor system 260 may implement the machine instructions stored in the memory system 250.

In an alternative embodiment, the communication interface 270 may allow the server system 200 to interface with the network 112. In this embodiment, the output system 230 may send communications to the communication interface 270. The communications system 275 communicatively links the output system 230, the input system 240, the memory system 250, the processor system 260 and/or the input/output system 280 to each other. The communications system 275 may include any one of, some of, any combination of, or all of one or more electrical cables, fiber optic cables, and/or sending signals through air or water (i.e., wireless communications). Some examples of sending signals through air and/or water may include systems for transmitting electromagnetic waves such as infrared and/or radio waves and/or systems for sending sound waves.

The input/output system 280 may include devices that have the dual function as the input and output devices. For example, the input/output system 280 may include one or more touch sensitive screens, which display an image and therefore may be an output device and accept input when the screens may be pressed by a finger or a stylus. The touch sensitive screens may be sensitive to heat and/or pressure. The input/output devices may be sensitive to a voltage or a current produced by a stylus. The input/output system 280 may be optional and may be utilized in addition to or in place of the output system 230 and/or the input device 240.

FIG. 3 illustrates a block diagram of a client system 300, in accordance with one embodiment of the present invention.

The client system 300 may include an output system 302, an input system 304, a memory system 306, a processor system 308, and a communications system 312, an input/output system 314, a website 316 and a wireless portal 318. Other embodiments of the client system 300 may not have all of the components and/or may have other embodiments in addition to or instead of the components listed above.

The client system 300 may be any one of the client systems 110, 114, 116, 118, 120 and/or handheld or mobile wireless device 122, SMARTPHONE® 124 or IPAD® 126 that may be utilized as one of the network devices of FIG. 3. In other embodiments, the client system 300 may include additional components and/or may not include all of the components listed above. The output system 302 may include any one of, some of, any combination of or all of a monitor system, a wireless transmitter, a handheld display system, a printer system, a speaker system, a connection or an interface system to a sound system, an interface system to peripheral devices and/or a connection and/or an interface system to a computer system, an intranet, and/or the Internet.

The input system 304 may include any one of, some of, any combination of or all of a keyboard system, a mouse system, a track ball system, a track pad system, one or more buttons on a handheld system, a scanner system, a wireless receiver, a microphone system, a connection to a sound system, and/or a connection and/or an interface system to a computer system, an intranet, and/or the Internet (i.e., Infrared Data Association or IrDA, Universal Serial Bus or USB).

The memory system 306 may include, any one of, some of, any combination of or all of a long-term storage system, such as a hard drive, a short term storage system, such as a random access memory; a removable storage system, such as a floppy drive or a removable drive and/or a flash memory. The memory system 306 may include one or more machine readable mediums that may store a variety of different types of information. The term machine readable medium may be utilized to refer to any medium that may be structurally configured for carrying information in a format that may be readable by a machine. One example of a machine-readable medium may be a computer-readable medium. The memory system 306 may store a non-transitory storage media for automatically recording a plurality of measurements and verifying rock specimens.

The processor system 308 may include any one of, some of, any combination of, or all of multiple parallel processors, a single processor, a system of processors having one or more central processors and/or one or more specialized processors dedicated to specific tasks. The processor system 308 may implement the programs stored in the memory system 306. The communications system 312 may communicatively link the output system 302, the input system 304, the memory system 306, the processor system 308, and/or the input/output system 314 to each other. The communications system 312 may include any one of, some of, any combination of, or all of one or more electrical cables, fiber optic cables, and/or sending signals through air or water (i.e., wireless communications). Some examples of sending signals through air and/or water may include systems for transmitting electromagnetic waves such as infrared and/or radio waves and/or systems for sending sound waves.

The input/output system 314 may include devices that have the dual function as input and output devices. For example, the input/output system 314 may include one or more touch sensitive screens, which display an image and therefore may be an output device and accept input when the screens may be pressed by a finger or a stylus. The touch sensitive screens may be sensitive to heat, capacitance and/or pressure. One or more of the input/output devices may be sensitive to a voltage or a current produced by a stylus. The input/output system 314 is optional, and may be utilized in addition to or in place of the output system 302 and/or the input device 304.

The client systems 110, 114, 116, 118, 120 and the handheld wireless device 122 may also be tied into a website 316 or a wireless portal 318 which may also be tied directly into the communications system 312. Any website 316 or wireless portal 318 may also include a non-transitory storage media and a website module (not shown) to maintain, allow access to and run the website as well.

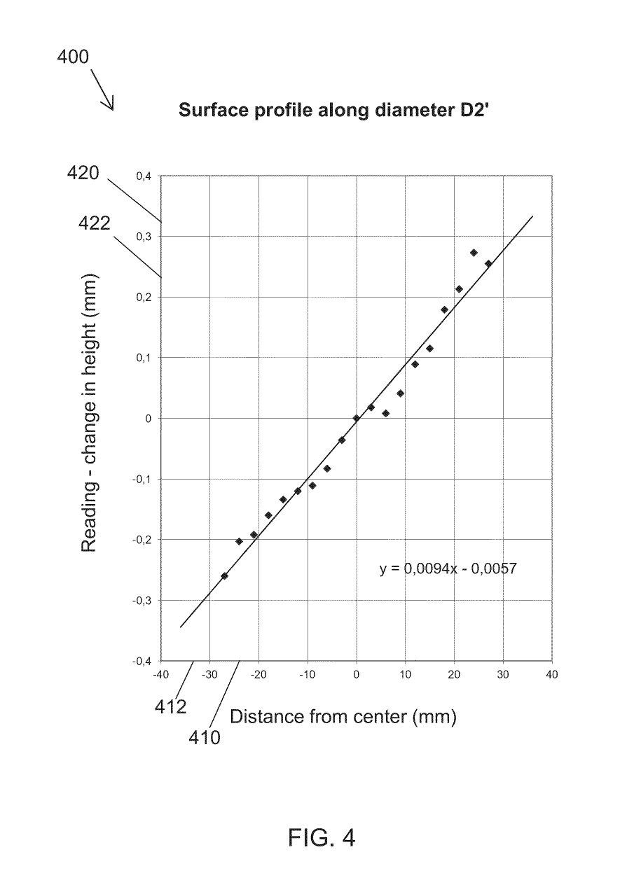

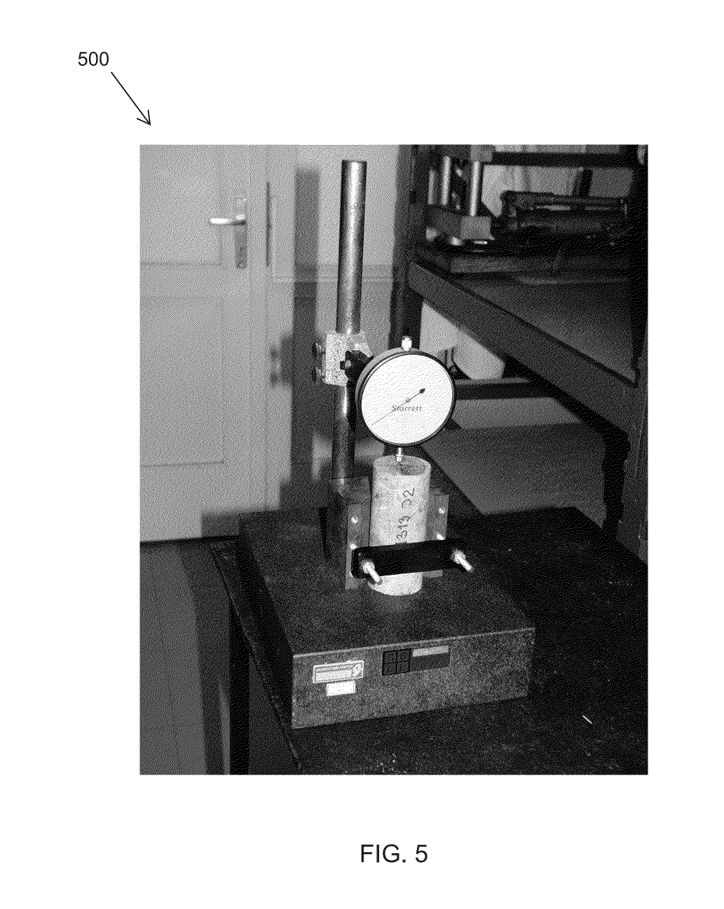

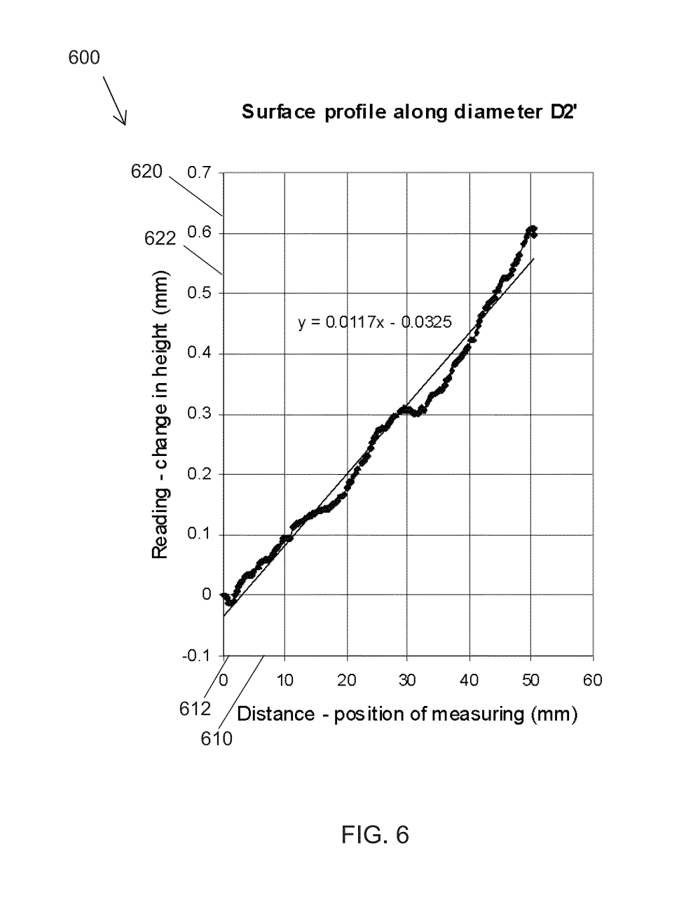

For the realization of the investigation goals of the system and method for automatic recording and verification of rock specimens, it may be necessary to ensure an effective and reliable recording of specimens, with minimal influence of human factor. The special equipment may be designed and proven in a laboratory as a coordinate table with an automatic data acquisition, for the recording of a plurality of surface profiles along chosen directions on sides and ends of a specimen. Results and equipment according to manual procedure described in ASTM D 4543, as illustrated in FIGS. 4 and 5, are replaced by the results obtained utilizing a device for automatic recording and verification of specimens, as illustrated in FIGS. 6, 7 and 8.

FIG. 4 illustrates a graph 400 of a manually recorded surface profile along a diameter, in accordance with one embodiment of the present invention.

The graph 400 includes an x-axis 410 and a y-axis 420. The x-axis 410 may be a distance from a center 412 in mms or other suitable dimensions. The y-axis 420 may be a change in height 422 in mms or other suitable dimensions.

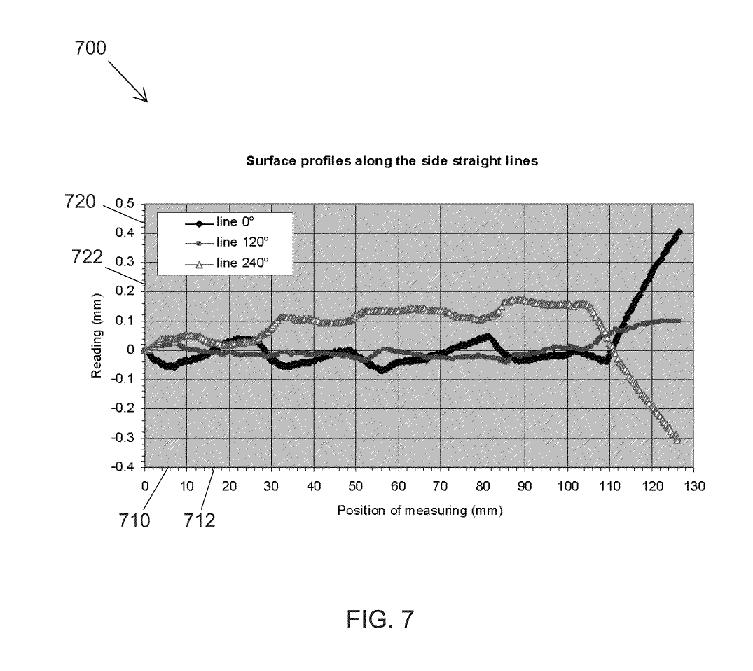

Discrete measurements at a distance intervals of approximately 3 mms are shown (from which the surface profile is approximated), for a diameter D2′ on the lower end (base) of the specimen (opposed to the diameter D2 on the upper end of the specimen). Instead of waviness on the specimen cylindrical surface (as in FIG. 7), usually the only known fact about the specimen cylindrical surface is whether a feeler gauge of nominal thickness of 0.5 mm passes or does not pass between the sample and the reference surface.

FIG. 5 illustrates a device 500 to manually record a surface profile along a diameter, in accordance with one embodiment of the present invention.

FIG. 6 illustrates a graph 600 of an automatically recorded surface profile along a diameter, in accordance with one embodiment of the present invention.

The graph 600 may include an x-axis 610 and a y-axis 620. The x-axis 610 may be a distance as a position of measuring 612 in mms or other suitable dimensions. The y-axis 620 may be a change in height 622 in mms or other suitable dimensions.

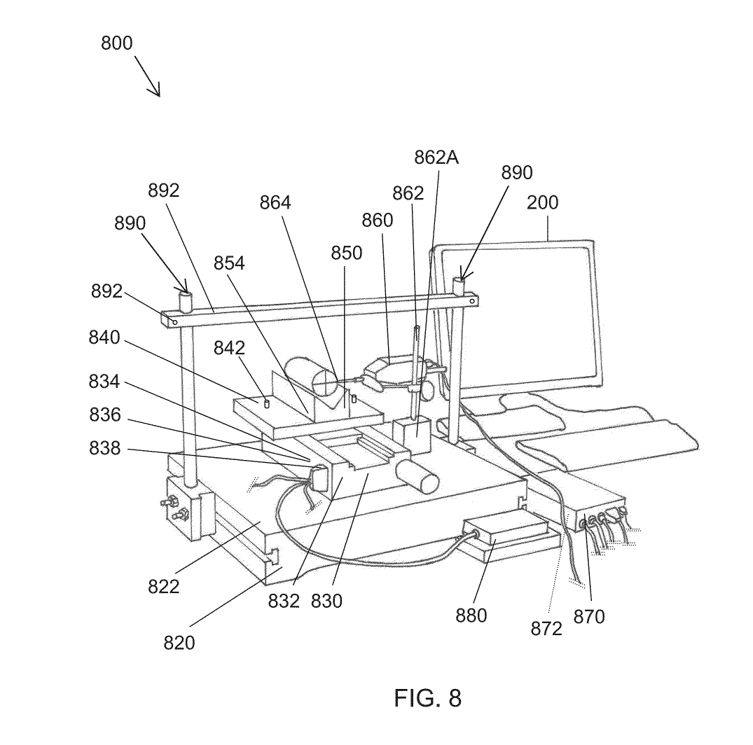

The surface profiles along the diameter (FIG. 6) and along the side straight lines on the specimen cylindrical surface (FIG. 7) were obtained using a device for automatic recording and verification of rock specimens (FIG. 8). In relation to FIG. 4, the difference is that the zeroing is performed at the beginning of the recording line (not relevant to the follow-up). In this sample, the flatness and perpendicularity along D2′ do not meet the ASTM requirements, while side surface satisfies the ASTM requirements.

FIG. 7 illustrates a graph 700 of a surface profiles along a plurality of straight lines on the specimen cylindrical surface, in accordance with one embodiment of the present invention.

The graph 700 may include an x-axis 710 and a y-axis 720. The x-axis 710 may be a position of measuring 712 in mms or other suitable dimensions. The y-axis 720 may be a reading (displacement) 722 in mms or other suitable dimensions.

FIG. 8 illustrates a system overview of a system 800 to automatically record a plurality of measurements and verify rock specimens, in accordance with one embodiment of the present invention.

The system 800 may be a component of a system (FIG. 1, 100) to automatically record a plurality of measurements and verify one or more rock specimens.

The system 800 may include a server system (FIG. 2, 200), a support surface 820, a motorized stage 830, a plate 840, a plurality of specimen holders as V-block 850, one or more digital dial gauges 860, a measurement system controller 870, a slide (motorized stage) motion controller 880 and a moving frame 890.

The server system 200 may include a memory system (FIG. 2, 250) and a rock specimens recording and verifying non-transitory storage media (FIG. 2, 255). The memory system 250 may be in communication with a processing system (FIG. 2, 260). The non-transitory storage media may reside on the memory system 250. The non-transitory storage media may include a plurality of rock specimens data 255. The support surface 820 may be a flat test surface 822 that allows working with the required flatness and accuracy. The motorized stage 830 may be disposed on the support surface 820. The motorized stage 830 (motorized linear translator, slide) may include a pair of sensors 832, a linear incremental encoder 834 as an optical sensor or other sensor 836 for determination of position of a plurality of measurements taken on rock specimens (FIG. 6, 610) along the axis of motorized linear slide 830 and a tachogenerator 838 for controlling speed of linear motion. The plate 840 may have a plurality of alignment pins 842. The V-block 850 with specimen may be configured by the alignment pins 842 or otherwise into a pair of orthogonal orientations of the support surface 820 (corresponding to the possible positions of specimen axis). The V-block 850 may be positioned for recording along a side straight line on the specimen cylindrical surface, or along a diameter of the specimen, a plurality of continuously recorded curves with a plurality of density (frequency) of surface readings at least ten times greater or greater than those in the standard ASTM D 4543. The V-block 850 may have a plurality of corresponding slots 854 for placement in two mutually perpendicular positions. The one or more digital dial gauges 860 or other displacement transducer with adequate measuring tip 864 to make a contact with the specimen surface may be fixed by means of a magnetic stand 862 or any other suitable method of fixturing. Optionally two or more displacement gauges or transducers with corresponding two or more means for mounting 862A may be utilized at the same time. In case of using two displacement devices, the two digital dial gauges or other transducers 860 may simultaneously record two opposing surface profiles along the specimen diameter or the side straight line on the specimen cylindrical surface, thereby generating a record of cross-section of the rock specimen. The measurement system controller 870 may include a plurality of control electronics 872 that electrically control the system 800. The slide motion controller 880 may move the motorized stage 830 forward and backward. The moving frame 890 may be mounted to slide along the support surface 820. The moving frame 890 may include a plurality of adaptors 892 as an alternative to magnetic stands to set one or more additional measuring devices.

The method for automatically recording a plurality of measurements and verification of rock specimens may include the steps of securing the rock specimens onto a V-block of a system to automatically record and verify rock specimens, actuating a linear motorized stage to collect a plurality of rock specimens data, sliding a digital dial gauge tip (by movement of the specimen) along one or more of a plurality of chosen surface lines, transmitting a plurality of chosen surface lines data to a server system and recording the chosen surface lines data.

The securing step may include the system which is independent of the way of sampling a plurality of specimen surface points and that may be in compliance with ASTM D4543 standard. The actuating step may include the linear motorized stage that may start constant speed linear motion. The sliding step may include the chosen surface lines that may be continuously recorded curves with a plurality of density readings at least ten times better or greater than those in the standard ASTM D 4543. The transmitting step may include the chosen surface lines data that may be repositioned for a subsequent data set. The recoding step may include the chosen surface lines data that may include at least seven data sets for each of the rock specimens (four diameters and three straight lines on the specimen cylindrical surface). The method may be executed by a non-transitory computer storage media having instructions stored thereon which, when executed, execute the method.

The system and method for automatically recording of a plurality of measurements and verification of rock specimens records the shape and the surface irregularities of one or more rock specimens with high resolution and accuracy. It is useful as additional equipment in laboratories for rock mechanics, or other suitable laboratories where the shape and surface regularities of the specimens are important. Desired result may be achieved by different levels of process automation. The rock specimens may be placed on a jig assembly in different ways. The method of probing a plurality of specimen surface points is independent from a method of fixturing of the specimen and transfer of measured data to a personal computer or other suitable device, which may be done in a number of ways other than an Ethernet interface.

The system and method produce continuously recorded curves with a plurality of density readings at least 10 times greater or greater than those in the standard ASTM D 4543. A trained operator in an hour may complete 5 to 6 full testing of specimens in contrast to the manual process, requiring approximately 1 hour/specimen. Except for research purposes, the system and method may also be utilized in laboratory work, enabling improved everyday reliability and significant time savings.

While the present invention has been related in terms of the foregoing embodiments, those skilled in the art will recognize that the present invention is not limited to the embodiments described. The present invention may be practiced with modification and alteration within the spirit and scope of the appended claims. Thus, the description is to be regarded as illustrative instead of restrictive on the present invention.

1. A system to automatically record a plurality of measurements and verify one or more rock specimens, comprising:
a server system with a processor system, a communications interface, a communications system, an input system and an output system, the server system having access to a communications network;
a memory system with an operating system, a communications module, a web browser module, a web server application and a rock specimens recording and verifying non-transitory storage media, the rock specimens recording and verifying non-transitory storage media resides on the memory system and includes a plurality of rock specimens data, the processor system is in communication with the memory system via the communications system;
a support surface with a flat test surface, the flat test surface allows obtaining a plurality of precise rock specimen data;
a motorized stage, with a motorized base or a motorized linear translator slide, the motorized stage disposed on the support surface, the motorized stage includes a pair of sensors, a linear incremental encoder for measuring position along an axis of motion and a tachogenerator for controlling speed of the motorized stage, the motorized stage performs movement and positioning of the one or more rock specimens along a chosen axis of motion;
a plate having a plurality of alignment pins;
a V-block configured by the alignment pins into a pair of orthogonal orientations of the support surface;
one or more digital dial gauges with a magnetic stand, the one or more digital dial gauges each having a digital dial gauge tip passing over a surface of the one or more rock specimens;
a measurement system controller with a plurality of control electronics, the measurement system controller electrically controls the system to automatically record a plurality of measurements and verify the one or more rock specimens;
a slide motion controller moving the motorized stage forward and backward; and
a moving frame mounted to slide along a side of the support surface above the flat test surface, the motorized stage, the plate, the V-block with the one or more rock specimens and the one or more digital dial gauges.

2. The system to automatically record a plurality of measurements and verify one or more rock specimens according to claim 1, further comprising a client system.

3. The system to automatically record a plurality of measurements and verify one or more rock specimens according to claim 2, wherein the client system is a personal computer.

4. The system to automatically record a plurality of measurements and verify one or more rock specimens according to claim 3, wherein the personal computer accesses the server system via the communications network.

5. The system to automatically record a plurality of measurements and verify one or more rock specimens according to claim 1, wherein the specimen surface is sampled and a surface profile is automatically recorded along a side straight line on a specimen cylindrical surface.

6. The system to automatically record a plurality of measurements and verify one or more rock specimens according to claim 5, wherein the specimen cylindrical surface is sampled and the surface profile is automatically recorded along a diameter on a specimen end.

7. The system to automatically record a plurality of measurements and verify one or more rock specimens according to claim 5, wherein the specimen surface is sampled and the surface profile is automatically registered as a plurality of continuously recorded curves.

8. The system to automatically record a plurality of measurements and verify one or more rock specimens according to claim 7, wherein the continuously recorded curves have a plurality of density readings at least ten times greater than standard ASTM D 4543.

9. The system to automatically record a plurality of measurements and verify one or more rock specimens according to claim 1, wherein the each digital dial gauge tip has a radius of curvature which corresponds to one or more surface irregularities to be recorded.

10. The system to automatically record a plurality of measurements and verify one or more rock specimens according to claim 1, wherein the moving frame includes a plurality of adaptors to connect one or more additional measuring devices.

11. A method for automatically recording a plurality of measurements and verification of one or more rock specimens, comprising the steps of:
securing the rock specimens onto a V-block of a system to automatically record a plurality of measurements and verify one or more rock specimens;
actuating a linear motorized stage to collect a plurality of rock specimens data;
sliding a digital dial gauge tip along one or more of a plurality of chosen lines at the same time on a rock specimen surface;
transmitting a plurality of chosen surface lines data to a server system; and
recording the chosen surface lines data on a memory system of the system to automatically record a plurality of measurements and verify one or more rock specimens.

12. The method according to claim 11, wherein the chosen surface lines are a plurality of continuously recorded curves with a plurality of density readings at least ten times greater than standard ASTM D 4543.

13. The method according to claim 11, wherein the chosen surface lines data are repositioned for a subsequent data set.

14. The method according to claim 11, wherein the chosen surface lines data include at least seven data sets for each of the one or more rock specimens.

15. The method according to claim 11, wherein the system to automatically record and verify the one or more rock specimens is in compliance with standard ASTM D4543.

16. A non-transitory computer storage media having instructions stored thereon which, when executed, execute a method comprising the steps of:
securing the rock mechanics specimens onto a V-block of a system to automatically record a plurality of measurements and verify one or more rock specimens;
actuating a linear motorized stage to collect a plurality of rock specimen's data;
sliding a digital dial gauge tip along one or more of a plurality of chosen lines at the same time on a rock specimen surface;
transmitting a plurality of chosen surface lines data to the system to automatically record a plurality of measurements and verify the one or more rock specimens; and
recording the chosen surface lines data on a memory system of the system to automatically record a plurality of measurements and verify one or more rock specimens.

17. The non-transitory computer storage media according to claim 16, wherein the system to automatically record and verify the one or more rock specimens is in compliance with standard ASTM D4543.

18. The non-transitory computer storage media according to claim 17, wherein the chosen surface lines are a plurality of continuously recorded curves with a plurality of density readings at least ten times greater than standard ASTM D 4543.

19. The non-transitory computer storage media according to claim 16, wherein the chosen surface lines data are repositioned for a subsequent data set.

20. The non-transitory computer storage media according to claim 16, wherein the chosen surface lines data include at least seven data sets for each of the one or more rock specimens.
